SEQUENCE LISTING (84 sequences)

SEQ ID NO: 1 - protein, 149 aa, Tityus serrulatus
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISKK

SEQ ID NO: 2 - protein, 234 aa, Tityus serrulatus
SPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 3 - protein, 15 aa, Tityus serrulatus
MFSLAYIILLATVSA

SEQ ID NO: 4 - protein, 383 aa, Tityus serrulatus
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISKKSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 5 - protein, 398 aa, Tityus serrulatus
MFSLAYIILLATVSAIPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISKKSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 6 - protein, 418 aa, synthetic construct
  VARIANT 165..184: note = Xaa may be present or absent and when present may be any amino acid
MFSLAYIILLATVSAIPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISKKXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 7 - protein, 403 aa, synthetic construct
  VARIANT 150..169: note = Xaa may be present or absent and when present may be any amino acid
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISKKXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 8 - protein, 417 aa, synthetic construct
  VARIANT 164..183: note = Xaa may be present or absent and when present may be any amino acid
MFSLAYIILLATVSAIPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISKXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 9 - protein, 402 aa, synthetic construct
  VARIANT 149..168: note = Xaa may be present or absent and when present may be any amino acid
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISKXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 10 - protein, 416 aa, synthetic construct
  VARIANT 163..182: note = Xaa may be present or absent and when present may be any amino acid
MFSLAYIILLATVSAIPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 11 - protein, 401 aa, synthetic construct
  VARIANT 148..167: note = Xaa may be present or absent and when present may be any amino acid
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKISXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 12 - protein, 415 aa, synthetic construct
  VARIANT 162..181: note = Xaa may be present or absent and when present may be any amino acid
MFSLAYIILLATVSAIPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKIXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 13 - protein, 400 aa, synthetic construct
  VARIANT 147..166: note = Xaa may be present or absent and when present may be any amino acid
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKIXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 14 - protein, 414 aa, synthetic construct
  VARIANT 161..180: note = Xaa may be present or absent and when present may be any amino acid
MFSLAYIILLATVSAIPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 15 - protein, 399 aa, synthetic construct
  VARIANT 146..165: note = Xaa may be present or absent and when present may be any amino acid
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVKXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 16 - protein, 413 aa, synthetic construct
  VARIANT 160..179: note = Xaa may be present or absent and when present may be any amino acid
MFSLAYIILLATVSAIPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 17 - protein, 398 aa, synthetic construct
  VARIANT 145..164: note = Xaa may be present or absent and when present may be any amino acid
IPSGRVDIVFPSLETSRSGVKIIKFEALGQDIELNLEPAGEILAKDFAIVDLNNQREHLTNVEDLKRKIYRDSVKGAALLIDENGPLTMQGIINSKLRIVPYESGRVIKDGRIAHQIVELINDEKSYINDVMPLDVNGVMENVVXXXXXXXXXXXXXXXXXXXXSPCIIIDYLCVTETTFTERFKTNKELLEYITVMFTGVQNLLDTLNLGIKAQVIGITPFKKQNEPSFIEDSAIPGHQQVLDPVDLVKNMAKYYCNNAKGLAKDADIIMLISNRKLGELQDDGTVAYNTAGISLGSGVCKQCSKVGVAQDDSDYNERVDTVAHETAHLIGAPHDEEGPEQTGISGSPGAKDCPESDGYIMGSGNNKVNKFKFSKCTKKCVEHLLSLPRASCVLADC

SEQ ID NO: 18 - protein, 141 aa, Tityus serrulatus
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMAR

SEQ ID NO: 19 - protein, 238 aa, Tityus serrulatus
DDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 20 - protein, 16 aa, Tityus serrulatus
MISYLASIFLLATVSA

SEQ ID NO: 21 - protein, 379 aa, Tityus serrulatus
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMARDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 22 - protein, 395 aa, Tityus serrulatus
MISYLASIFLLATVSAVPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMARDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 23 - protein, 415 aa, synthetic construct
  VARIANT 158..177: note = Xaa may be present or absent and when present may be any amino acid
MISYLASIFLLATVSAVPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMARXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 24 - protein, 399 aa, synthetic construct
  VARIANT 142..161: note = Xaa may be present or absent and when present may be any amino acid
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMARXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 25 - protein, 414 aa, synthetic construct
  VARIANT 157..176: note = Xaa may be present or absent and when present may be any amino acid
MISYLASIFLLATVSAVPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMAXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 26 - protein, 398 aa, synthetic construct
  VARIANT 141..160: note = Xaa may be present or absent and when present may be any amino acid
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMAXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 27 - protein, 413 aa, synthetic construct
  VARIANT 156..175: note = Xaa may be present or absent and when present may be any amino acid
MISYLASIFLLATVSAVPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 28 - protein, 397 aa, synthetic construct
  VARIANT 140..159: note = Xaa may be present or absent and when present may be any amino acid
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRMXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 29 - protein, 412 aa, synthetic construct
  VARIANT 155..174: note = Xaa may be present or absent and when present may be any amino acid
MISYLASIFLLATVSAVPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 30 - protein, 396 aa, synthetic construct
  VARIANT 139..158: note = Xaa may be present or absent and when present may be any amino acid
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSRXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 31 - protein, 411 aa, synthetic construct
  VARIANT 154..173: note = Xaa may be present or absent and when present may be any amino acid
MISYLASIFLLATVSAVPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 32 - protein, 395 aa, synthetic construct
  VARIANT 138..157: note = Xaa may be present or absent and when present may be any amino acid
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVSXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 33 - protein, 410 aa, synthetic construct
  VARIANT 153..172: note = Xaa may be present or absent and when present may be any amino acid
MISYLASIFLLATVSAVPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 34 - protein, 394 aa, synthetic construct
  VARIANT 137..156: note = Xaa may be present or absent and when present may be any amino acid
VPSGRVEVVFPSVETSRSGVKTVKFTALDQDVELKLRSAGEILGKRFAIQDVDVESLRRKIYRDSVNGAALLIDEDGPLTIEGIVNSKLRIQPFESGRITKDGIIAHQIVEVIDDKKSYDRVAVIPENVKRNAENVXXXXXXXXXXXXXXXXXXXXDDDCIVVEYYIVTDSAFTKRFKSNSALTNYVTVMFTGVQNLMDTLELGIGVRLLGVTTFTEKTEPSFIKDNLIPGPPAAFDPDVLISAMSKYYCNHQTGLAKDTDLIFLITARGMGDPREDGTVDINTAGIANSAGVCKPCFKSGIATDDSDYNERVDTLAHESVHLLGSPHDGEGPNLVSLEGSPGAANCPAKAGYIMGNRNDKVNKYKFSNCTKKCVEYLLSKPTASCIFQQCSDF

SEQ ID NO: 35 - protein, 138 aa, Centruroides sculpturatus
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMAR

SEQ ID NO: 36 - protein, 235 aa, Centruroides sculpturatus
KDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 37 - protein, 16 aa, Centruroides sculpturatus
MENLYFQGIPSGRVDV

SEQ ID NO: 38 - protein, 373 aa, Centruroides sculpturatus
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMARKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 39 - protein, 389 aa, Centruroides sculpturatus
MENLYFQGIPSGRVDVVFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMARKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 40 - protein, 409 aa, synthetic construct
  VARIANT 155..174: note = Xaa may be present or absent and when present may be any amino acid
MENLYFQGIPSGRVDVVFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMARXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 41 - protein, 393 aa, synthetic construct
  VARIANT 139..158: note = Xaa may be present or absent and when present may be any amino acid
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMARXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 42 - protein, 408 aa, synthetic construct
  VARIANT 154..173: note = Xaa may be present or absent and when present may be any amino acid
MENLYFQGIPSGRVDVVFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMAXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 43 - protein, 392 aa, synthetic construct
  VARIANT 138..157: note = Xaa may be present or absent and when present may be any amino acid
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMAXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 44 - protein, 407 aa, synthetic construct
  VARIANT 153..172: note = Xaa may be present or absent and when present may be any amino acid
MENLYFQGIPSGRVDVVFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 45 - protein, 391 aa, synthetic construct
  VARIANT 137..156: note = Xaa may be present or absent and when present may be any amino acid
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATMXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 46 - protein, 406 aa, synthetic construct
  VARIANT 152..171: note = Xaa may be present or absent and when present may be any amino acid
MENLYFQGIPSGRVDVVFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 47 - protein, 390 aa, synthetic construct
  VARIANT 136..155: note = Xaa may be present or absent and when present may be any amino acid
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVATXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 48 - protein, 346 aa, synthetic construct
  VARIANT 151..170: note = Xaa may be present or absent and when present may be any amino acid
MENLYFQGIPSGRVDVVFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVAXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEP

SEQ ID NO: 49 - protein, 389 aa, synthetic construct
  VARIANT 135..154: note = Xaa may be present or absent and when present may be any amino acid
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVAXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 50 - protein, 404 aa, synthetic construct
  VARIANT 150..169: note = Xaa may be present or absent and when present may be any amino acid
MENLYFQGIPSGRVDVVFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 51 - protein, 388 aa, synthetic construct
  VARIANT 134..153: note = Xaa may be present or absent and when present may be any amino acid
VFPSVETSRSGVKTVKFRALNEEIELKLEPAGEILAKNFALVNQQPNSIDVEDLKRRIYRDSTNGAALLIDEDGPVTIQGIVNSKFRIEPYESGRTVKDGQVPHKIVEVINDEKSFLNDRVVHMDVNEEMEKVXXXXXXXXXXXXXXXXXXXXKDQCIVVECLVVTESAFTKRFETTKALTEYVTVMYTGVQNLIDTLQLGIKFRLLGIDPFTKETEPPYIEESANPVNPKYLNPLDLIDRMGKYYCNHATGLAKDADMIMLLVTRNLGELKDDGTVKFRVVGLAYKGAVCKQCYKVGVCKDDSYYNERVDTVAHESAHLLGSPHDGEPGAANLVSLGTKDCPASKDGYIMGNRRDKVNKYKFSKCTKKCVKDALSLPEAKCVYESCG

SEQ ID NO: 52 - protein, 153 aa, Mesobuthus eupeus
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIQS

SEQ ID NO: 53 - protein, 236 aa, Mesobuthus eupeus
DECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 54 - protein, 13 aa, Mesobuthus eupeus
MLVHLLVVLFFVA

SEQ ID NO: 55 - protein, 389 aa, Mesobuthus eupeus
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIQSDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 56 - protein, 402 aa, Mesobuthus eupeus
MLVHLLVVLFFVAVGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIQSDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 57 - protein, 422 aa, synthetic construct
  VARIANT 167..186: note = Xaa may be present or absent and when present may be any amino acid
MLVHLLVVLFFVAVGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIQSXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 58 - protein, 409 aa, synthetic construct
  VARIANT 154..173: note = Xaa may be present or absent and when present may be any amino acid
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIQSXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 59 - protein, 421 aa, synthetic construct
  VARIANT 166..185: note = Xaa may be present or absent and when present may be any amino acid
MLVHLLVVLFFVAVGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIQXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 60 - protein, 408 aa, synthetic construct
  VARIANT 153..172: note = Xaa may be present or absent and when present may be any amino acid
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIQXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 61 - protein, 420 aa, synthetic construct
  VARIANT 165..184: note = Xaa may be present or absent and when present may be any amino acid
MLVHLLVVLFFVAVGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 62 - protein, 407 aa, synthetic construct
  VARIANT 152..171: note = Xaa may be present or absent and when present may be any amino acid
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGIXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 63 - protein, 419 aa, synthetic construct
  VARIANT 164..183: note = Xaa may be present or absent and when present may be any amino acid
MLVHLLVVLFFVAVGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 64 - protein, 406 aa, synthetic construct
  VARIANT 151..170: note = Xaa may be present or absent and when present may be any amino acid
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAGXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 65 - protein, 418 aa, synthetic construct
  VARIANT 163..182: note = Xaa may be present or absent and when present may be any amino acid
MLVHLLVVLFFVAVGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 66 - protein, 405 aa, synthetic construct
  VARIANT 150..179: note = Xaa may be present or absent and when present may be any amino acid
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTAXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 67 - protein, 417 aa, synthetic construct
  VARIANT 162..181: note = Xaa may be present or absent and when present may be any amino acid
MLVHLLVVLFFVAVGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 68 - protein, 404 aa, synthetic construct
  VARIANT 149..168: note = Xaa may be present or absent and when present may be any amino acid
VGAIPSGRIDVVYPSMETLRSGMKSIRLRALDQDLELRLEPAGDVIADDFTIVHGENGEKDHTFNVQNLKRKLYRDVEKGSALHIDEDGPLTINGILNSELRIEPDTSEKRSENGIIAHRVIEVLKDENLRYDIATIPPGAIRSVNYTXXXXXXXXXXXXXXXXXXXXDECIQVEYLFVAESSFTEMLSKTAKKIRGRVVEVYLANLMNAVKMLFNTLDLRIKVVIIGIIIHTKANQANLGPYIPSQPNGLAAEETLKKMAKFYCKSTDILVLRADIVMLITKRPLGDYDEKKKFSDNTAGIAFPNTVCRKCYKYGLVTDDSKLNERADTVAHESAHLLGCDHDGEGDERTGSKDCPASDGYIMGDRNKINGMKFSPCCKRSVRKQLQVAESRCIIEDCDDIKV

SEQ ID NO: 69 - protein, 4 aa, synthetic construct
IEGR

SEQ ID NO: 70 - protein, 4 aa, synthetic construct
IDGR

SEQ ID NO: 71 - protein, 6 aa, synthetic construct
LVPRGS

SEQ ID NO: 72 - protein, 7 aa, synthetic construct
ENLYFQG

SEQ ID NO: 73 - protein, 7 aa, synthetic construct
ENLYFQS

SEQ ID NO: 74 - protein, 8 aa, synthetic construct
LEVLFQGP

SEQ ID NO: 75 - protein, 5 aa, synthetic construct
DDDDK

SEQ ID NO: 76 - DNA, 1194 bp, synthetic construct
  source 1..1194: mol_type = other DNA
atgtttagcctggcgtatattattctgctggcgaccgtgagcgcgattccgagcggccgcgtggatattgtgtttccgagcctggaaaccagccgcagcggcgtgaaaattattaaatttgaagcgctgggccaggatattgaactgaacctggaaccggcgggcgaaattctggcgaaagattttgcgattgtggatctgaacaaccagcgcgaacatctgaccaacgtggaagatctgaaacgcaaaatttatcgcgatagcgtgaaaggcgcggcgctgctgattgatgaaaacggcccgctgaccatgcagggcattattaacagcaaactgcgcattgtgccgtatgaaagcggccgcgtgattaaagatggccgcattgcgcatcagattgtggaactgattaacgatgaaaaaagctatattaacgatgtgatgccgctggatgtgaacggcgtgatggaaaacgtggtgaaaattagcaaaaaaagcccgtgcattattattgattatctgtgcgtgaccgaaaccacctttaccgaacgctttaaaaccaacaaagaactgctggaatatattaccgtgatgtttaccggcgtgcagaacctgctggataccctgaacctgggcattaaagcgcaggtgattggcattaccccgtttaaaaaacagaacgaaccgagctttattgaagatagcgcgattccgggccatcagcaggtgctggatccggtggatctggtgaaaaacatggcgaaatattattgcaacaacgcgaaaggcctggcgaaagatgcggatattattatgctgattagcaaccgcaaactgggcgaactgcaggatgatggcaccgtggcgtataacaccgcgggcattagcctgggcagcggcgtgtgcaaacagtgcagcaaagtgggcgtggcgcaggatgatagcgattataacgaacgcgtggataccgtggcgcatgaaaccgcgcatctgattggcgcgccgcatgatgaagaaggcccggaacagaccggcattagcggcagcccgggcgcgaaagattgcccggaaagcgatggctatattatgggcagcggcaacaacaaagtgaacaaatttaaatttagcaaatgcaccaaaaaatgcgtggaacatctgctgagcctgccgcgcgcgagctgcgtgctggcggattgc

SEQ ID NO: 77 - DNA, 1185 bp, synthetic construct
  source 1..1185: mol_type = other DNA
atgattagctatctggcgagcatttttctgctggcgaccgtgagcgcggtgccgagcggccgcgtggaagtggtgtttccgagcgtggaaaccagccgcagcggcgtgaaaaccgtgaaatttaccgcgctggatcaggatgtggaactgaaactgcgcagcgcgggcgaaattctgggcaaacgctttgcgattcaggatgtggatgtggaaagcctgcgccgcaaaatttatcgcgatagcgtgaacggcgcggcgctgctgattgatgaagatggcccgctgaccattgaaggcattgtgaacagcaaactgcgcattcagccgtttgaaagcggccgcattaccaaagatggcattattgcgcatcagattgtggaagtgattgatgataaaaaaagctatgatcgcgtggcggtgattccggaaaacgtgaaacgcaacgcggaaaacgtgagccgcatggcgcgcgatgatgattgcattgtggtggaatattatattgtgaccgatagcgcgtttaccaaacgctttaaaagcaacagcgcgctgaccaactatgtgaccgtgatgtttaccggcgtgcagaacctgatggataccctggaactgggcattggcgtgcgcctgctgggcgtgaccacctttaccgaaaaaaccgaaccgagctttattaaagataacctgattccgggcccgccggcggcgtttgatccggatgtgctgattagcgcgatgagcaaatattattgcaaccatcagaccggcctggcgaaagataccgatctgatttttctgattaccgcgcgcggcatgggcgatccgcgcgaagatggcaccgtggatattaacaccgcgggcattgcgaacagcgcgggcgtgtgcaaaccgtgctttaaaagcggcattgcgaccgatgatagcgattataacgaacgcgtggataccctggcgcatgaaagcgtgcatctgctgggcagcccgcatgatggcgaaggcccgaacctggtgagcctggaaggcagcccgggcgcggcgaactgcccggcgaaagcgggctatattatgggcaaccgcaacgataaagtgaacaaatataaatttagcaactgcaccaaaaaatgcgtggaatatctgctgagcaaaccgaccgcgagctgcatttttcagcagtgcagcgatttt

SEQ ID NO: 78 - DNA, 1167 bp, synthetic construct
  source 1..1167: mol_type = other DNA
atggaaaacctgtattttcagggcattccgagcggccgcgtggatgtggtgtttccgagcgtggaaaccagccgcagcggcgtgaaaaccgtgaaatttcgcgcgctgaacgaagaaattgaactgaaactggaaccggcgggcgaaattctggcgaaaaactttgcgctggtgaaccagcagccgaacagcattgatgtggaagatctgaaacgccgcatttatcgcgatagcaccaacggcgcggcgctgctgattgatgaagatggcccggtgaccattcagggcattgtgaacagcaaatttcgcattgaaccgtatgaaagcggccgcaccgtgaaagatggccaggtgccgcataaaattgtggaagtgattaacgatgaaaaaagctttctgaacgatcgcgtggtgcatatggatgtgaacgaagaaatggaaaaagtggcgaccatggcgcgcaaagatcagtgcattgtggtggaatgcctggtggtgaccgaaagcgcgtttaccaaacgctttgaaaccaccaaagcgctgaccgaatatgtgaccgtgatgtataccggcgtgcagaacctgattgataccctgcagctgggcattaaatttcgcctgctgggcattgatccgtttaccaaagaaaccgaaccgccgtatattgaagaaagcgcgaacccggtgaacccgaaatatctgaacccgctggatctgattgatcgcatgggcaaatattattgcaaccatgcgaccggcctggcgaaagatgcggatatgattatgctgctggtgacccgcaacctgggcgaactgaaagatgatggcaccgtgaaatttcgcgtggtgggcctggcgtataaaggcgcggtgtgcaaacagtgctataaagtgggcgtgtgcaaagatgatagctattataacgaacgcgtggataccgtggcgcatgaaagcgcgcatctgctgggcagcccgcatgatggcgaaccgggcgcggcgaacctggtgagcctgggcaccaaagattgcccggcgagcaaagatggctatattatgggcaaccgccgcgataaagtgaacaaatataaatttagcaaatgcaccaaaaaatgcgtgaaagatgcgctgagcctgccggaagcgaaatgcgtgtatgaaagctgcggc

SEQ ID NO: 79 - DNA, 1206 bp, synthetic construct
  source 1..1206: mol_type = other DNA
atgctggtgcatctgctggtggtgctgttttttgtggcggtgggcgcgattccgagcggccgcattgatgtggtgtatccgagcatggaaaccctgcgcagcggcatgaaaagcattcgcctgcgcgcgctggatcaggatctggaactgcgcctggaaccggcgggcgatgtgattgcggatgattttaccattgtgcatggcgaaaacggcgaaaaagatcatacctttaacgtgcagaacctgaaacgcaaactgtatcgcgatgtggaaaaaggcagcgcgctgcatattgatgaagatggcccgctgaccattaacggcattctgaacagcgaactgcgcattgaaccggataccagcgaaaaacgcagcgaaaacggcattattgcgcatcgcgtgattgaagtgctgaaagatgaaaacctgcgctatgatattgcgaccattccgccgggcgcgattcgcagcgtgaactataccgcgggcattcagagcgatgaatgcattcaggtggaatatctgtttgtggcggaaagcagctttaccgaaatgctgagcaaaaccgcgaaaaaaattcgcggccgcgtggtggaagtgtatctggcgaacctgatgaacgcggtgaaaatgctgtttaacaccctggatctgcgcattaaagtggtgattattggcattattattcataccaaagcgaaccaggcgaacctgggcccgtatattccgagccagccgaacggcctggcggcggaagaaaccctgaaaaaaatggcgaaattttattgcaaaagcaccgatattctggtgctgcgcgcggatattgtgatgctgattaccaaacgcccgctgggcgattatgatgaaaaaaaaaaatttagcgataacaccgcgggcattgcgtttccgaacaccgtgtgccgcaaatgctataaatatggcctggtgaccgatgatagcaaactgaacgaacgcgcggataccgtggcgcatgaaagcgcgcatctgctgggctgcgatcatgatggcgaaggcgatgaacgcaccggcagcaaagattgcccggcgagcgatggctatattatgggcgatcgcaacaaaattaacggcatgaaatttagcccgtgctgcaaacgcagcgtgcgcaaacagctgcaggtggcggaaagccgctgcattattgaagattgcgatgatattaaagtg

SEQ ID NO: 80 - protein, 6 aa, synthetic construct
HHHHHH

SEQ ID NO: 81 - protein, 130 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRG

SEQ ID NO: 82 - protein, 153 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGT

SEQ ID NO: 83 - protein, 366 aa, Escherichia coli
KIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 84 - protein, 26 aa, Escherichia coli
KRRWKKNFIAVSAANRFKKISSSGAL

(19) United States
(12) Patent Application Publication
(10) Pub. No.: US 2025/0101398 A1
(43) Pub. Date: Mar. 27, 2025

(54) FAST ACTING SNARE-CLEAVING ENZYMES

(71) Applicants: Paul L. FLETCHER, Greenville, NC (US); Maryann FLETCHER, Greenville, NC (US)

(72) Inventors: Paul L. FLETCHER, Greenville, NC (US); Maryann FLETCHER, Greenville, NC (US)

(21) Appl. No.: 18/729,703

(22) PCT Filed: Jan. 17, 2023
(86) PCT No.: PCT/US2023/060731

§ 371 Date: Jul. 17, 2024

Related U.S. Application Data
(60) Provisional application No. 63/300,458, filed on Jan. 18, 2022.

(51) Int. Cl. C12N 9/64; A61K 38/00

(52) U.S. Cl. CPC C12N 9/6421; A61K 38/00; C07K 2319/02; C07K 2319/50

(57) ABSTRACT

The present invention relates to polypeptides that may act to cleave SNARE proteins. In particular, the present invention relates to proteins that include a catalytic polypeptide attached to a prodomain polypeptide, optionally through a linker (e.g., an enzyme cleavable linker). Methods of using such polypeptides to treat disorders are also provided herein.

### STATEMENT OF PRIORITY

This application claims the benefit, under 35 U.S.C. § 119 (e), of U.S. Provisional Application No. 63/300,458 filed on Jan. 18, 2022, the entire contents of which is incorporated by reference herein.

### STATEMENT REGARDING ELECTRONIC FILING OF A SEQUENCE LISTING

A Sequence Listing in XML format, entitled 1597-2WO_ST26.xml, 123,254 bytes in size, generated on Jan. 17, 2023 and filed herewith, is hereby incorporated by reference into the specification for its disclosures.

### FIELD OF THE INVENTION

The present invention provides metalloprotease enzymes isolated from scorpion venom that cleave SNARE complex proteins, their nucleic acid and amino acid sequences, and methods of use thereof in the treatment of various diseases, disorders, and cosmetic conditions.

### BACKGROUND OF THE INVENTION

Eukaryotic intracompartmental transport and secretory processes require fusion of vesicles with cellular membranes. A step leading to fusion of vesicles with cellular membranes is assembly of a tetrameric coiled-coil structure formed from sets of membrane proteins known as SNAREs (soluble N-ethyl maleimide sensitive factor attachment protein receptors. The final, post-fusion ternary SNARE complex combines one vesicle protein (v-SNARE) with two target membrane proteins (t-SNARES). The SNAREs responsible for neuronal secretion are among the best-studied examples of this family of proteins. They include the v-SNARE VAMP (vesicle-associated membrane protein) that is located on the secretory vesicle membrane and two t-SNAREs, syntaxin (sx) and SNAP25 (synaptosomal-associated protein 25), present on the target membrane.

SNAREs are responsible for selective transport between cellular compartments. Alterations or damage to proteins or membranes that perform integral transport functions often produce disabling or irreversible consequences. Failure of the normal vesicular traffic (unless transient), which is the basis for intracellular transport and secretion, results in development of disease. As a consequence, agents that attack this machinery have significant effects on normal cellular processes.

### SUMMARY

Provided according to embodiments of the invention are recombinant polypeptides that include a prodomain polypeptide linked to a catalytic polypeptide. In some embodiments, the catalytic polypeptide, or a biologically active fragment thereof, has an activity of proteolytic cleavage of synaptosomal nerve-associated protein 25 (SNAP25), vesicle-associated membrane protein 2 (VAMP2), vesicle-associated membrane protein 8 (VAMP8), vesicle-associated membrane protein 7 (VAMP7), and/or other soluble N-ethyl maleimide sensitive factor attachment protein receptors (SNARE) proteins.

In some embodiments, a catalytic polypeptide comprises an amino acid sequence of any one of SEQ ID NO:2 (Antarease I), SEQ ID NO:19 (Antarease II), SEQ ID NO:36 (Azantarease I), SEQ ID NO:53 (MeVP), and/or a biologically active fragment of any of the foregoing. In some embodiments, a biologically active fragment is a sequence of SEQ ID NO:2, SEQ ID NO:19, SEQ ID NO:36, and/or SEQ ID NO:53 (MeVP) with one or more of the amino acid residues located (e.g., 1, 2, 3, 4, or 5) at the N-terminus of the catalytic polypeptide absent.

In some embodiments, a prodomain polypeptide comprises an amino acid sequence of any one of SEQ ID NO:1 (Antarease I), SEQ ID NO:18 (Antarease II), SEQ ID NO:35 (Azantarease I), SEQ ID NO:52 (MeVP), and/or a biological active fragment of any of the forgoing. In some embodiments, a biologically active fragment is an amino acid sequence of any one of SEQ ID NO:1, SEQ ID NO:18, SEQ ID NO:35, and/or SEQ ID NO:52 (MeVP) with one or more of the amino acid residues (e.g., 1, 2, 3, 4, 5, 6, 7, 8, 9, or 10) located at the C-terminus of the prodomain polypeptide absent.

In some embodiments, a recombinant polypeptide comprises the prodomain polypeptide of SEQ ID NO:1 (or biologically active fragment thereof) and the catalytic polypeptide of SEQ ID NO:2 (or a biologically active fragment thereof), (e.g., SEQ ID NO:4 or a biologically active fragment thereof) (Antarease I). In some embodiments, a recombinant polypeptide comprises the prodomain polypeptide of SEQ ID NO:18 (or biologically active fragment thereof) and the catalytic polypeptide of SEQ ID NO:19 (or biologically active fragment thereof), (e.g., SEQ ID NO:21 or biologically active fragment thereof) (Antarease II). In some embodiments, a recombinant polypeptide comprises the prodomain polypeptide of SEQ ID NO:35 (or biologically active fragment thereof) and the catalytic polypeptide of SEQ ID NO:36 (or biologically active fragment thereof), (e.g., SEQ ID NO:38 or biologically active fragment thereof) (Azantarease I). In some embodiments, a recombinant polypeptide comprises the prodomain polypeptide of SEQ ID NO:52 (or biologically active fragment thereof) and the catalytic polypeptide of SEQ ID NO:53 (or biologically active fragment thereof), (e.g., SEQ ID NO:55 or biologically active fragment thereof) (MeVP).

In some embodiments, the inventors have created novel proteins by linking prodomain polypeptides with catalytic peptides using a cleavable linker. In some embodiments, a recombinant polypeptide comprises a linker attached to the C-terminus of a prodomain polypeptide and to the N-terminus of a catalytic polypeptide. Such recombinant polypeptides may have the general structure of.
Prodomain Polypeptide-Linker-Catalytic Polypeptide

In some embodiments, a recombinant polypeptide comprising a linker may comprise the amino acid sequence of any one of SEQ ID NO:6, SEQ ID NO:7, SEQ ID NO:23, SEQ ID NO:24, SEQ ID NO:40, SEQ ID NO:41, SEQ ID NO:57, and SEQ ID NO:58.

The present invention further provides a nucleic acid construct encoding a recombinant polypeptide of the invention. Also provided is a vector comprising one or more nucleic acid constructs of the invention (e.g., nucleic acid constructs encoding one or more of the recombinant polypeptides of the invention).

In other aspects of the invention, a composition is provided comprising a recombinant polypeptide of the present invention in a pharmaceutically acceptable carrier.

In some embodiments, provided are methods of using a composition of the present invention to treat or prevent certain diseases and disorders or to reduce the risk of developing such diseases and disorders. In some embodiments, provided are methods of using a composition of the invention to treat or prevent a cosmetic condition or to reduce the risk of developing a cosmetic condition in a subject. In particular embodiments, the compositions of the invention may be used to treat cystic fibrosis.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Except as otherwise indicated, standard methods known to those skilled in the art may be used for cloning genes, amplifying, and detecting nucleic acids, and the like. Such techniques are known to those skilled in the art. See, e.g., Sambrook et al., Molecular Cloning: A Laboratory Manual 2nd Ed. (Cold Spring Harbor, NY, 1989); Ausubel et al. Current Protocols in Molecular Biology (Green Publishing Associates, Inc. and John Wiley & Sons, Inc., New York).

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

Unless otherwise defined, the term “at least one” has the same meaning as “one or more” (e.g., 1, 2, 3, 4, 5 and the like).

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In some embodiments, the reduction can result in no or essentially no (i.e., an insignificant amount, e.g., less than about 10% or even 5%) detectable activity or amount.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, for example, a nucleic acid molecule and/or a nucleotide sequence may express a polypeptide of interest.

A “heterologous” or a “recombinant” nucleotide sequence is a nucleotide sequence not naturally associated with a host cell into which it is introduced, including non-naturally occurring multiple copies of a naturally occurring nucleotide sequence.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length (e.g., reduced by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950 or 1000 or more nucleotides or any range or value therein) relative to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent.

In some embodiments, a nucleic acid fragment or portion (or region) may comprise, consist essentially of or consist of about 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390,395, 400, 420, 440, 460, 480, 500, 520, 540, 560, 580, 600, 620, 640, 660, 680, 690, 700, 750, 800, 850, 900, 950, or 1000, or more consecutive nucleotides of a nucleotide sequence of the invention. As used herein with respect to nucleic acids, the term “functional fragment” refers to nucleic acid that encodes a functional fragment of a polypeptide.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 260, 270, 280, 290, 300, or 350, or more consecutive amino acids of a reference polypeptide (e.g., a polypeptide of the invention). As used herein with respect to polypeptides, the term “biologically active fragment” refers to a fragment of a polypeptide of the invention that has SNARE-cleaving activity.

A “region” of a polynucleotide or a polypeptide refers to a portion of consecutive nucleotides or consecutive amino acid residues of that polynucleotide or a polypeptide, respectively.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in in-frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity, e.g., substantial complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection.

In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more nucleotides in length, and any range therein, up to the full length of the sequence. In some embodiments, nucleotide sequences can be substantially identical over at least about 20 consecutive nucleotides (e.g., about 20, 21, 22, 23, 24, 25, 26, 27, 28, 29, 30, 31, 32, 33, 34, 35, 36, 37, 38, 39, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2500, 3000, 3500, 4000 or more nucleotides).

In some embodiments of the invention, the substantial identity exists over a region of consecutive amino acid residues of a polypeptide of the invention that is about 3 amino acid residues to about 10, 11, 12, 13, 14, 15, 16, 17, 18, 19, or 20 amino acid residues, about 5 amino acid residues to about 25, 30, 35, 40, 45, 50 or 60 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8, 9, 10, 11, 12, 13, 14, or more consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 275, 300, 325, or 350, or more amino acids in length or more consecutive amino acid residues In some embodiments, a substantially identical nucleotide or protein sequence may perform substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4 6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

A polynucleotide or nucleic acid construct of the invention may be operatively associated with a variety of promoters and/or other regulatory elements for expression. Thus, in some embodiments, a polynucleotide or nucleic acid construct of this invention may further comprise one or more promoters, introns, enhancers, and/or terminators operably linked to one or more nucleotide sequences.

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker. The term “linker” is art-recognized and refers to a chemical group, or a molecule linking two molecules or moieties, e.g., two domains of a fusion protein, such as, for example, a catalytic domain and a prodomain. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid, or it may be a peptide. In some embodiments, the linker is a peptide. In some embodiments, a linker may be a cleavable linker as described herein.

In some embodiments, a peptide linker useful with this invention may be about 2 to about 50 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids in length (e.g., about 2 to about 10, about 2 to about 20, about 2 to about 30, about 4 to about 40, about 4 to about 50, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, or more amino acids in length (see, e.g., SEQ ID NO:69 (Factor Xa linker), SEQ ID NO:70 (Factor Xa linker), SEQ ID NO:71 (Thrombin linker), SEQ ID NO:72 (TEV linker), SEQ ID NO:73 (TEV linker), SEQ ID NO:74 (Prescission Protease linker), and SEQ ID NO:75 (Enterokinase linker)).

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence. In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression, e.g., a promoter region. These include a TATA box consensus sequence, and often a CAAT box consensus sequence (Breathnach and Chambon, (1981) Annu. Rev. Biochem. 50:349). Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, expression control sequences, and/or selectable markers useful in selecting, measuring, and monitoring nucleic acid transfer results (e.g., delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J Biol. Chem. 263:14621 (1988); and Hartmut et al., Canadian Patent Application No. 2,012,311, filed Mar. 15, 1990).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. In representative embodiments, transfection is directed to particular cell types in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931). It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J Biol. Chem. 262:4429 (1987)).

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

As used herein, the term “biological sample” refers to a sample that contains or consists of material obtained or derived from an organism, e.g., a mammal, e.g., a human, non-human primate, rodent (e.g., mouse, rabbit, rat), dog, cat, or other domesticated animal or bird, etc. A biological sample can comprise, for example, any of the following: whole blood, fractionated blood, plasma, serum, other blood fraction or blood product, aqueous humor, vitreous humor, tears, synovial fluid, cerebrospinal fluid, saliva, mucous, sweat, milk, urine, fecal material, pericardial fluid, gastric fluid, peritoneal fluid, pleural fluid, cyst fluid, broncheolar lavage fluid, nasal lavage, lymphatic fluid, mammary fluid, duct fluid, tears, tissue extract, glandular secretion, tissue extract, whole cell lysate or fraction thereof, cell or tissue extract, tissue homogenate, or cell culture supernatant.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage).

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a cell in such a manner that the nucleotide sequence gains access to the interior of a cell.

Proteolytic cleavage of SNARE family components was originally known to be associated only with large microbial proteins from the genus Clostridium. These clostridial proteins require intracellular cleavage before they are released as smaller active proteolytic enzymes.

The present inventors have previously discovered that certain metalloproteases isolated from scorpion venom that can cleave SNARE complex proteins. See, U.S. Pat. No. 9,149,666, which is incorporated herein by reference in its entirety. However, while these catalytic metalloproteases isolated from scorpion venom can cleave SNARE complex proteins in vitro, there has been difficulty in obtaining activity in vivo. As such, new polypeptides, compositions, nucleic acids, and methods have been developed, as will be discussed in further detail below.

Provided according to embodiments of the invention is a recombinant polypeptide comprising a prodomain polypeptide linked to a catalytic polypeptide. In some embodiments, the catalytic polypeptide, or a biologically active fragment thereof, has an activity of proteolytic cleavage of synaptosomal nerve-associated protein 25 (SNAP25), vesicle-associated membrane protein 2 (VAMP2), vesicle-associated membrane protein 8 (VAMP8), vesicle-associated membrane protein 7 (VAMP7), and/or other soluble N-ethyl maleimide sensitive factor attachment protein receptors (SNARE) proteins.

In some embodiments, a prodomain, a polypeptide sequence attached to the N-terminus of the catalytic protein, is needed for the recombinant polypeptide to have the proper folding and/or structure to be active or substantially active in vivo. In some embodiments, the prodomain may be cleaved in vivo to produce the catalytic polypeptide, or a biologically active fragment thereof, that can act to cleave a SNARE protein such as SNAP25, VAMP2, and/or VAMP8. Catalytic polypeptides have been identified from the venom of certain scorpion species. Antarease I and II proteins have been isolated from venom of the South American scorpion Tityus serrulatus (TSV), and Azantarease I has been isolated from the venom of a North American scorpion species, Centruroides sculpturatus (CSV). MeVP, or Venom metalloprotease-2 is isolated from venom of the scorpion Mesobuthus eupeus. These enzymes have been discovered to have SNARE cleaving activity. In addition, the prodomain sequences for these catalytic proteins have now been identified so that certain polypeptides of the invention have the general structure of:
Prodomain Polypeptide-Catalytic Polypeptide

The prodomain and catalytic polypeptide sequences are the wild-type sequences. In some embodiments, a catalytic polypeptide comprises an amino acid sequence of any one of SEQ ID NO:2 (Antarease I), SEQ ID NO:19 (Antarease II), SEQ ID NO:36 (Azantarease I), SEQ ID NO:53 (MeVP), and/or a biologically active fragment of any of the foregoing. In some embodiments, a biologically active fragment is a sequence of SEQ ID NO:2, SEQ ID NO:19, SEQ ID NO:36, and/or SEQ ID NO:53 (MeVP) with one or more of the amino acid residues located (e.g., 1, 2, 3, 4, or 5) at the N-terminus of the catalytic polypeptide absent.

In some embodiments, a prodomain polypeptide comprises an amino acid sequence of any one of SEQ ID NO:1 (Antarease I), SEQ ID NO:18 (Antarease II), SEQ ID NO:35 (Azantarease I), SEQ ID NO:52 (MeVP), and/or a biological active fragment of any of the forgoing. In some embodiments, a biologically active fragment is an amino acid sequence of any one of SEQ ID NO:1, SEQ ID NO:18, SEQ ID NO:35, and/or SEQ ID NO:52 (MeVP) with one or more of the amino acid residues (e.g., 1, 2, 3, 4, 5, 6, 7, 8, 9, or 10) located at the C-terminus of the prodomain polypeptide absent.

In some embodiments, a recombinant polypeptide comprises the prodomain polypeptide of SEQ ID NO:1 (or biologically active fragment thereof) and the catalytic polypeptide of SEQ ID NO:2 (or a biologically active fragment thereof), (e.g., SEQ ID NO:4 or a biologically active fragment thereof) (Antarease I). In some embodiments, a recombinant polypeptide comprises the prodomain polypeptide of SEQ ID NO:18 (or biologically active fragment thereof) and the catalytic polypeptide of SEQ ID NO:19 (or biologically active fragment thereof), (e.g., SEQ ID NO:21 or biologically active fragment thereof) (Antarease II). In some embodiments, a recombinant polypeptide comprises the prodomain polypeptide of SEQ ID NO:35 (or biologically active fragment thereof) and the catalytic polypeptide of SEQ ID NO:36 (or biologically active fragment thereof), (e.g., SEQ ID NO:38 or biologically active fragment thereof) (Azantarease I). In some embodiments, a recombinant polypeptide comprises the prodomain polypeptide of SEQ ID NO:52 (or biologically active fragment thereof) and the catalytic polypeptide of SEQ ID NO:53 (or biologically active fragment thereof), (e.g., SEQ ID NO:55 or biologically active fragment thereof) (MeVP).

In some embodiments, a recombinant polypeptide may further comprise a signal peptide attached to the N-terminus of the prodomain polypeptide so that the recombinant polypeptide has the general structure of.
Signal peptide-Prodomain Polypeptide-Catalytic Polypeptide

In some embodiments, a signal peptide may comprise the amino acid sequence of any one of SEQ ID NO:3, SEQ ID NO:20, SEQ ID NO:37, and SEQ ID NO:54. Thus, in some embodiments, a recombinant polypeptide comprising a signal polypeptide may comprise any one of SEQ ID NO:5 (Antarease I), SEQ ID NO:22 (Antarease II), SEQ ID NO:39 (Azantarease I), or SEQ ID NO:56 (MeVP).

In some embodiments, the inventors have created novel proteins by linking prodomain polypeptides with catalytic peptides as described herein using a linker (e.g., a cleavable linker). In some embodiments, a recombinant polypeptide comprises a linker attached to the C-terminus of a prodomain polypeptide and to the N-terminus of a catalytic polypeptide. Such recombinant polypeptides may have the general structure of:
Prodomain Polypeptide-Linker-Catalytic Polypeptide

The prodomain may facilitate the activity of the polypeptides in vivo. However, it may also be desirable for the prodomain to be removed in vivo during the SNARE protein cleaving process. Accordingly, when the linker is cleavable (e.g., by an enzyme), the prodomain may be removed in vivo, which may promote SNARE protein cleavage by the catalytic protein. In some embodiments, a recombinant polypeptide comprising a linker may comprise the amino acid sequence of any one of SEQ ID NO:6, SEQ ID NO:7, SEQ ID NO:23, SEQ ID NO:24, SEQ ID NO:40, SEQ ID NO:41, SEQ ID NO:57, and SEQ ID NO:58.

In some embodiments of the invention, a linker may comprise about 1 to 20 amino acids, including 1-10 amino acids (e.g., 1, 2, 3, 4, 5, 6, 7, 8, 9, or 10 amino acids and any range defined therein).

In some embodiments, a linker may replace a portion (e.g., 1-20 amino acid residues) of the amino acid residues at the C-terminus of the prodomain polypeptide and/or a portion (e.g., 1-20 amino acid residues) of the amino acid residues at the N-terminus of the catalytic protein. In some embodiments, a linker may replace at least one amino acid residue of the last ten amino acids located at the C-terminus of the prodomain polypeptide to which it is linked. Thus, for example, 1, 2, 3, 4, 5, 6, 7, 8, 9, or 10 of the last ten amino acid residues located at the C-terminus of the prodomain polypeptide may be absent. Examples of recombinant polypeptides in which one or more amino acid residues at the C-terminus of the prodomain is absent when a linker is present include, but are not limited to, the any one of SEQ ID NOS:8-17 (Antarease I); SEQ ID NOS:25-34 (Antarease II); SEQ ID NOS:42-51 (Azantarease I); and SEQ ID NOS:59-68 (MeVP). In some embodiments, in addition to or in lieu of the removal of certain amino acid residues at the C-terminus of the prodomain polypeptide, 1, 2, 3, 4, 5, 6, 7, 8, 9, or 10 of the amino acids at the N-terminus of the catalytic polypeptide may be absent.

In some embodiments, a recombinant polypeptide may include an amino acid linker (peptide linker) that is cleavable by an enzyme (e.g., a protease). In some embodiments, a linker may have the peptide sequence of any one of SEQ ID NO:69 (Factor Xa linker), SEQ ID NO:70 (Factor Xa linker), SEQ ID NO:71 (Thrombin linker), SEQ ID NO:72 (TEV linker), SEQ ID NO:73 (TEV linker), SEQ ID NO:74 (Prescission Protease linker), or SEQ ID NO:75 (Enterokinase linker), but other cleavable linkers may be used.

In some embodiments, a recombinant polypeptide may comprise a purification or affinity tag, optionally wherein the purification or affinity tag may be attached to the N-terminus of the prodomain polypeptide or to the N-terminus of the signal peptide. Any purification tag known in the art may be used, including but not limited to, His-Tag (e.g., SEQ ID NO:80), Trx-His Tag (e.g., SEQ ID NO:81 or SEQ ID NO:82), a Maltose Binding Protein (MBP; e.g., SEQ ID NO:83), and/or a Calmodulin Binding protein (CBP; e.g., SEQ ID NO:84). In some embodiments, there may be a linker between the affinity or purification tag and the N-terminus of the prodomain polypeptide or the N-terminus of the signal peptide. In some embodiments, the linker may include one or more enzyme cleavable linkers described herein, e.g., SEQ ID NO:69 (Factor Xa linker), SEQ ID NO:70 (Factor Xa linker), SEQ ID NO:71 (Thrombin linker), SEQ ID NO:72 (TEV linker), SEQ ID NO:73 (TEV linker), SEQ ID NO:74 (Prescission Protease linker), or SEQ ID NO:75 (Enterokinase linker).

In some embodiments, the polypeptides of the invention may be configured to bind to a targeting moiety so that the peptide may bind (e.g., preferentially bind) to a biological site of interest, such as a SNARE protein (e.g., SNAP25, VAMP2, VAMP7, and/or VAMP8).

The present invention further provides a nucleic acid construct encoding a recombinant polypeptide of the invention. Nucleic acid constructs of this invention can comprise a nucleotide sequence that can be identical in sequence or, due to the well-characterized degeneracy of the nucleic acid code, can include alternative codons that encode the same amino acid. Furthermore, nucleic acids of this invention can comprise nucleotide sequences that can include codons which represent conservative substitutions of amino acids as are well known in the art, such that the biological activity of the resulting polypeptide and/or fragment is retained.

In some embodiments of the invention, a recombinant polypeptide may be encoded by any one of the nucleotide sequences of SEQ ID NO:76 (Antarease I), SEQ ID NO:77 (Antarease II), SEQ ID NO:78 (Azantarease I), or SEQ ID NO:79 (MeVP). Such sequences may code for a polypeptide according to an embodiment of the invention, including polypeptides having activity of proteolytic cleavage of SNAP25, VAMP2, VAMP8, VAMP7, and/or other SNARE protein.

Accordingly, in some aspects of the present invention, nucleic acids encoding the recombinant polypeptides of the invention are provided. In some embodiments, a nucleic acid comprises a nucleotide sequence having at least about 70% (e.g., at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) sequence identity to any one of SEQ ID NO:76, SEQ ID NO:77, SEQ ID NO:78, or SEQ ID NO:79, or encodes an amino acid sequence having at least about 70% sequence identity to any one of SEQ ID NOs:1-68.

Accordingly, in some aspects of the present invention, nucleic acids encoding the recombinant polypeptides of the invention are provided. In some embodiments, a nucleic acid comprises a nucleotide sequence having at least about 70% (e.g., at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) sequence identity to any one of SEQ ID NO:76, SEQ ID NO:77, SEQ ID NO:78, or SEQ ID NO:79, or encodes an amino acid sequence having at least about 70% sequence identity to any one of SEQ ID NOs:6-17, 23-34, 40-51, and 57-68.

In some embodiments, a nucleic acid construct is provided, where the nucleic acid construct comprises a nucleotide sequence: (a) encoding the amino acid sequence of any one of SEQ ID NOS:1-68, (b) comprises a sequence having at least 70% sequence identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) to (a) above; and/or (c) comprises a nucleotide sequence that differs from the nucleotide sequences of (a) or (b) above due to the degeneracy of the genetic code.

In some embodiments, a nucleic acid construct is provided, where the nucleic acid construct comprises a nucleotide sequence: (a) encoding the amino acid sequence of any one of SEQ ID NOs:6-17, 23-34, 40-51, 57-68, (b) comprises a sequence having at least 70% sequence identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) to (a) above; and/or (c) comprises a nucleotide sequence that differs from the nucleotide sequences of (a) or (b) above due to the degeneracy of the genetic code.

Also provided is a vector comprising one or more nucleic acid constructs of the invention (e.g., nucleic acid constructs encoding one or more of the recombinant polypeptides of the invention).

In other aspects of the invention, a composition is provided comprising a recombinant polypeptide of the present invention in a pharmaceutically acceptable carrier. “Pharmaceutically acceptable,” as used herein, means a material that is not biologically or otherwise undesirable, i.e., the material may be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; latest edition). Exemplary pharmaceutically acceptable carriers for the compositions of this invention include, but are not limited to, sterile pyrogen-free water and sterile pyrogen-free physiological saline solution, human serum albumin, NaCl, lactose, sucrose, glycerol, alcohol, propylene glycol, fatty alcohols, triglycerides, fatty acid esters, and/or mineral oils.

Also provided is a composition comprising a recombinant polypeptide of the invention in a cosmetically acceptable carrier. The phrase “cosmetically acceptable carrier”, as used herein, means any substantially non-toxic carrier suitable for topical administration to the skin, which has suitable aesthetic properties and is compatible with the active agent of the present invention. By “compatible” it is meant that the active agent will remain stable and retain substantial activity therein. Suitable topical cosmetically acceptable carriers include, but are not limited to, water, petroleum jelly, petrolatum, mineral oil, vegetable oil, animal oil, organic and inorganic waxes, such as microcrystalline, paraffin and ozocerite wax, natural polymers, such as xanthanes, gelatin, cellulose, collagen, starch or gum arabic, synthetic polymers, alcohols, polyols, and the like.

Preferably, because of its non-toxic topical properties, the pharmaceutically and/or cosmetically-acceptable carrier is substantially miscible in water. Such water miscible carrier compositions can also include sustained or delayed release carriers, such as liposomes, microsponges, microspheres or microcapsules, aqueous based ointments, water-in-oil or oil-in-water emulsions, gels and the like.

Formulations suitable for topical administration include those for medical use and use in personal care and/or hygiene (e.g., soaps, skin creams, soaps, cleansers, shampoos). Topical compositions can include the active agents with vitamin E, vitamin A, conjugated linoleic acid, and essential fatty acids. The topical compositions disclosed herein are suitable for topical application to mammalian skin. The compositions comprise a safe and effective amount of the active agents, and a cosmetically and/or pharmaceutically acceptable topical carrier.

Thus, in some embodiments, the present invention provides a composition comprising, consisting essentially of and/or consisting of a recombinant polypeptide and/or nucleic acid of this invention in a pharmaceutically acceptable carrier and/or a cosmetically acceptable carrier and, optionally, further comprising other medicinal agents, pharmaceutical agents, stabilizing agents, buffers, carriers, adjuvants, diluents, etc. In some embodiments, the compositions of the present invention comprise a safe and effective amount of the active agents, and a cosmetically acceptable carrier.

In some embodiments, a composition of the invention may be in the form of a spray, an emulsion, a mousse, a liquid, a cream, an oil, a lotion, an ointment, a gel or a solid (including e.g., detergent-like substances such as BioPorter). For injection, a carrier will typically be a liquid. For other methods of administration, a carrier may generally be either solid or liquid. For inhalation administration, a carrier will be respirable, optionally in solid or liquid particulate form (e.g., powder).

In some embodiments, the compositions of the present invention can be useful in treatment or prevention of diseases and disorders or reduction of the risk of developing diseases and disorders. In some embodiments, the compositions of the invention are useful for treatment of or prevention of or reducing the risk of developing a cosmetic condition in a subject. In some embodiments, a method is provided for treating/preventing or reducing the risk of developing spasmodic muscles in a subject, the method comprising administering to the subject in need thereof an effective amount of a composition of the present invention as described herein.

In some embodiments, a composition as described herein, can be used to treat/prevent/reduce the risk of developing diseases or disorders including, without limitation, strabismus (crossed or misaligned eyes), blepharospasm (eyelid spasms), hemifacial spasm (unilateral muscle contractions of the face), headache pain including pain due to a migraine headache, cervical dystonia, primary axillary hyperhidrosis (excessive underarm sweating), palmar hyperhidrosis, prostatic symptoms, asthma, stroke symptoms including but not limited to post stroke spasticity, diabetes, obstructive pulmonary disease, achalasia (esophageal motility disorder), chronic obstructive pulmonary disease (COPD), back pain, cerebral palsy including but not limited to pediatric spastic cerebral palsy, chronic anal fissure, delayed gastric emptying, dysphonia including but not limited to spasmodic dysphonia and/or oromandibular dysphonia, epilepsy, epiphora, esotropia, essential tremor, eye lift, facial myokemia, fibromyalgia, flushing, Grey's syndrome, musculoskeletal pain syndromes, pancreatitis, Parkinson's disease, puborectalis syndrome, rhinitis, sialorrhea, tardive dyskinesia, tennis elbow, Tourette's syndrome, urinary incontinence, vaginismus, writer's cramp, laryngeal dystonia, lingual dystonia, cervical dystonia, focal hand dystonia, blepharospasm, anismus, hemifacial spasm, focal spasticity, spasmodic colitis, neurogenic bladder, limb spasticity, tics, bruxism, dysphagia, lacrimation, excessive salivation, and/or excessive gastrointestinal secretions, as well as other secretory disorders, pain from muscle spasms and the like (see, Cheng et al. Amer. J. Health-Syst. Pharm. 63:145-152 (2006) and U.S. Pat. No. 6,908,925).

Thus, in some embodiments, a method of treating and/or preventing a disorder and/or reducing the risk of developing in a subject is provided, comprising administering to the subject an effective amount of a composition as described herein, wherein the disorder may include, but is not limited to, strabismus (crossed or misaligned eyes), blepharospasm (eyelid spasms), hemifacial spasm (unilateral muscle contractions of the face), headaches including migraine headaches, cervical dystonia, primary axillary hyperhidrosis (excessive underarm sweating), palmar hyperhidrosis, prostatic symptoms including but not limited to benign prostatic hyperplasia, asthma, stroke symptoms, diabetes, obstructive pulmonary disease, achalasia (esophageal motility disorder), chronic obstructive pulmonary disease (COPD), back pain, cerebral palsy including but not limited to pediatric spastic cerebral palsy, chronic anal fissure, delayed gastric emptying, dysphonia including but not limited to spasmodic dysphonia and/or oromandibular dysphonia, epilepsy, epiphora, esotropia, essential tremor, eye lift, facial myokemia, fibromyalgia, flushing, Grey's syndrome, musculoskeletal pain syndromes, pancreatitis, Parkinson's disease, puborectalis syndrome, rhinitis, sialorrhea, tardive dyskinesia, tennis elbow, Tourette's syndrome, urinary incontinence, vaginismus, writer's cramp, laryngeal dystonia, cystic fibrosis, lingual dystonia, cervical dystonia, focal hand dystonia, blepharospasm, anismus, hemifacial spasm, focal spasticity, spasmodic colitis, neurogenic bladder, limb spasticity, tics, bruxism, dysphagia, lacrimation, excessive salivation, and/or excessive gastrointestinal secretions, as well as other secretory disorders, and pain from muscle spasms. In particular embodiments, the disorder is cystic fibrosis.

In other aspects of the invention, cosmetic conditions for which the compositions of the present invention can be useful to treat or prevent or reduce the risk of developing include, but are not limited to, the prevention and/or treatment of wrinkles and fine lines in the skin of a subject. Such treatments of the skin of a subject include without limitation the treatment of the face and neck. In some embodiments of the invention, such treatments include, but are not limited to, frown wrinkles, forehead wrinkles, lateral canthal wrinkles (“crow's feet”), brow furrows, nose crease wrinkles, wrinkles around the mouth and ears. In some embodiments, the treatment includes treatment of the cutaneous skin tissue of the subject. In some embodiments, the treatment includes treatment of the subcutaneous skin tissue of the subject. Treatments of the skin of the face and neck as described above of minimize of appearance of, for example, frown wrinkles, forehead wrinkles, lateral canthal wrinkles (“crow's feet”), brow furrows, nose crease wrinkles, wrinkles around the mouth and ears, over time (e.g., days, weeks, months, years) as compared to the absence of treatment with (absence of administration of) the compositions of the present invention.

A “subject” of this invention includes any subject that is susceptible to the various diseases, disorders and/or cosmetic conditions described herein. Nonlimiting examples of subjects of this invention include mammals, such as humans, nonhuman primates, domesticated mammals (e.g., dogs, cats, rabbits, guinea pigs, rats), livestock and agricultural mammals (e.g., horses, bovine, pigs, goats). In other embodiments, a subject may additionally be an animal such as a bird or reptile. Thus, in some embodiments, a subject can be any domestic, commercially or clinically valuable animal. Subjects may be male or female and may be any age including neonate, infant, juvenile, adolescent, adult, and geriatric subjects. In particular embodiments, the subject is a human. A human subject of this invention can be of any age, gender, race or ethnic group (e.g., Caucasian (white), Asian, African, black, African American, African European, Hispanic, Mideastern, etc.).

A subject of this invention can be “in need of” the methods of the present invention, e.g., because the subject has, or is believed at risk for, muscle spasms and/or another disease, disorder and/or cosmetic condition including those described herein and/or is a subject that would benefit from the methods of this invention. In some embodiments, a subject in need of the methods of this invention can be, but is not limited to, a subject diagnosed with, having or suspected to have, or at risk of having or developing any of the disorders and diseases described herein. For example, a subject in need of the methods of this invention can be, but is not limited to, a subject diagnosed with, having or suspected to have, or at risk of having or developing strabismus (crossed eyes). In another example, a subject in need of the methods of this invention can be, but is not limited to, a subject diagnosed with, having or suspected to have, or at risk of having or developing cystic fibrosis.

A further aspect of the invention is a method of administering or delivering a recombinant polypeptide of the invention, a fragment thereof, and/or a nucleic acid encoding the same, to a subject of this invention. Administration or delivery to a human subject or an animal in need thereof can be by any means known in the art for administering polypeptides, protein fragments and/or nucleic acids. In some embodiments, a polypeptide, fragment thereof and/or nucleic acid is delivered in a therapeutically effective dose in a pharmaceutically acceptable carrier.

In embodiments in which a nucleic acid of this invention is delivered in a viral vector (e.g., a virus particle), the dosage of virus particles to be administered to a subject will depend upon the mode of administration, the disease or condition to be treated, the individual subject's condition, the particular virus vector, and the nucleic acid to be delivered, and can be determined in a routine manner. Exemplary doses are virus titers of at least about 10⁵, 10⁶, 10⁷, 10⁸, 10⁹, 10¹⁰, 10¹¹, 10¹², 10³, 10¹⁴, 10¹⁵ transducing units or more, preferably about 10⁸-10¹³ transducing units, yet more preferably 10¹² transducing units.

Additional non-limiting exemplary modes of administration of the recombinant proteins, nucleic acids and vectors of this invention can include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to facial, neck, skeletal, diaphragm and/or cardiac muscle], intradermal, intrapleural, intracerebral, and intraarticular), topical (e.g., to both skin and mucosal surfaces, including airway surfaces, and transdermal administration, and the like, as well as direct tissue or organ injection (e.g., to liver, skeletal muscle, cardiac muscle, diaphragm muscle or brain). Administration can also be to a tumor (e.g., in or a near a tumor or a lymph node). The most suitable route in any given case will depend on the nature and severity of the condition being treated and on the nature of the particular protein, peptide, fragment, nucleic acid or vector that is being used.

Dosages of the recombinant polypeptides and/or active fragments thereof and/or nucleic acids encoding the same, to be administered to a subject will depend upon the mode of administration, the disease or condition to be treated, the individual subject's condition including, but not limited to, age and weight, the particular polypeptide and/or active fragment and/or nucleic acid encoding same, and any other agents being administered to the subject and can be determined in a routine manner according to methods well known in the art. In some embodiments, an exemplary dosage range is from about 0.001 unit to about 10,000 units. In other embodiments, the dosage range can be from about 100 units to about 10,000 units. In still other embodiments, the dosage range can be from about 0.01 unit to about 5000 units. In yet further embodiments, the dosage range can be from about 1 unit to about 5000 units. In some embodiments, the dosage range can be from about 10 units to about 1000 units. In other embodiments, the dosage range can be from about 100 units to about 500 units. In still other embodiments, the dosage range can be from about 20 units to about 100 units. Units are LD₅₀ units determined using 20-30 g Swiss-Webster mice (see, e.g., U.S. Pat. Nos. 7,494,661 and 7,491,403)

Although examples of routes of administration and dosages are provided, the appropriate route of administration and dosage are generally determined on a case-by-case basis by the attending physician. Such determinations are routine to one of ordinary skill in the art (see for example, Harrison's Principles of Internal Medicine (1998), edited by Anthony Fauci et al., McGraw Hill 14th edition).

In particular embodiments, more than one administration (e.g., two, three, four or more administrations) of a recombinant polypeptide, biologically active fragment and/or nucleic acid of this invention may be employed to achieve the desired result over a period of various intervals, e.g., daily, weekly, monthly, yearly, etc.

The present invention will now be described with reference to the following examples. It should be appreciated that these examples are for the purpose of illustrating aspects of the present invention, and do not limit the scope of the invention as defined by the claims.

### EXAMPLES

### Example 1: Example Protein Production and Purification Method

(1) For protein overexpression, plasmids are transformed in Escherichia coli Origami-2 (DE3) cells (Novagen), are grown in Luria-Bertani medium supplemented with 30 μgmL-1 kanamycin. After initial growth of the culture at 37° C. to an OD600 nm≈0.6, the culture is cooled to 18° C. and protein expression induced with 0.4 mM isopropyl-β-D-1-thiogalactopyranoside for 18-20 h.

(2) Pelleted cells from the cultures are washed in buffer A (50 mM Tris-HCl, 500 mM NaCl, 20 mM imidazole, pH7.4), harvested, and resuspended in buffer A, which contain EDTA-free Protease Inhibitor Cocktail tablets (Roche Applied Science) and DNAse I (Roche Applied Science).

(3) Cells are broken with a cell disrupter (Constant Cell Disruption Systems or French Pressure Cell) at a pressure of 1.35 kbar and non-disrupted cells and cell debris are removed by centrifugation at 35,000×g for 45 min. The supernatant is filtered (0.22 μm pore size; Millipore) and incubated at 4° C. for 1 h with nickel-nitrilotriacetic acid resin (Invitrogen) previously equilibrated with buffer A.

(4) Subsequently, the sample is applied to a batch purification open column (BioRad) and washed with five column volumes of buffer A. The affinity (e.g., His6)-tagged protein is eluted with buffer A containing 250 mM imidazole.

(5) The protein sample is dialyzed overnight at 4° C. against buffer A in the presence of 1 mM 1,4-dithio-DL-threitol (Sigma) and subsequently digested at room temperature for 24 h with affinity-tagged tobacco-etch virus protease at an enzyme:substrate ratio of 1:200 (w/w). The digested sample is again passed through the nickel-nitrilotriacetic acid resin column previously equilibrated with buffer A.

(6) The eluate is collected, concentrated by ultrafiltration, and further purified by size-exclusion chromatography with a Superdex 75 10-300 GL column (GE Healthcare) previously equilibrated with buffer B (20 mM Tris-HCl, 150 mM NaCl, pH7.4).

(7) Active protein is obtained by limited tryptic digestion of purified protein at an enzyme:substrate ratio of 1:100 (w/w) at room temperature for 3 h in buffer B.

(8) After trypsin inactivation with 1 mg mL-1 Pefabloc® SC (Roche), the sample is dialyzed against buffer C (20 mM Tris-HCl, 40 mM NaCl, pH7.4) overnight at 4° C. and subjected to anion-exchange chromatography to remove the inactivated serine protease in a MonoQ HR5/50 column (GE Healthcare) equilibrated with buffer C.

(9) Elution is carried out by using a linear gradient from 40 to 250 mM NaCl within 30 column volumes. Protein-containing fractions were pooled, concentrated by ultrafiltration, and subjected to a final size-exclusion chromatography step with a Superdex 75 10-300 GL column equilibrated with buffer B.

At all stages of purification, the purity of the protein samples is assessed by 14% SDS-PAGE (Laemmli buffer) stained with Coomassie blue. All ultrafiltration steps are done with Vivaspin 500 and Vivaspin 2 filter devices of 5-kDa cut-off (Sartorius Stedim Biotech or Amicon ultrafilter). The concentration of the proteins is determined with the BCA protein assay kit (Thermo Scientific) by using bovine serum albumin as a standard. Concentrated protein samples are stored at 4° C.

### Example 2

The catalytic peptide sequences of Antarease I, Antarease II, and Azantarease I were shown to have SNARE-cleaving activity, as described in U.S. Pat. No. 9,149,666, which is incorporated herein by reference in its entirety. However, it was subsequently discovered that the catalytic polypeptide, although active in vitro, was not sufficiently active in vivo. As such, in the present invention, the inventors have determined that by including a prodomain polypeptide linked to the catalytic polypeptide, optionally through a linker (e.g., an enzyme cleavable linker), the activity of the proteins may be improved in vivo. Without being bound to any theory, it is believed that the prodomain acts as a chaperone and the residues that follow are in some way bound to yield a functional conformation and/or other important chaperones present in the endoplasmic reticulum (ER) are attracted to associate with the polypeptide with the prodomain and yield the final functional conformation of the catalytic domain.

The foregoing is illustrative of the present invention and is not to be construed as limiting thereof. Instead, the definition is defined by the following claims, and equivalents thereof.

We claim:

1. A recombinant polypeptide comprising a prodomain polypeptide linked to a catalytic polypeptide, wherein the catalytic polypeptide, or a biologically active fragment thereof, has an activity of proteolytic cleavage of SNAP25, VAMP2, VAMP7, and/or VAMP8.

2. The recombinant polypeptide of claim 1, wherein the prodomain polypeptide comprises an amino acid sequence selected from the group consisting of SEQ ID NO:1, SEQ ID NO:18, SEQ ID NO:35, SEQ ID NO:52, and a biologically active fragment of any of the foregoing, and/or the catalytic polypeptide comprises an amino acid sequence selected from the group consisting of SEQ ID NO:2, SEQ ID NO:19, SEQ ID NO:36, SEQ ID NO: 53, and a biologically active fragment of any of the foregoing.

3. The recombinant polypeptide of claim 2, wherein the prodomain polypeptide comprises the amino acid sequence of SEQ ID NO:1, or a biologically active fragment thereof, and the catalytic polypeptide comprises the amino acid sequence of SEQ ID NO:2, or a biologically active fragment thereof.

4. The recombinant polypeptide of claim 2, wherein the prodomain polypeptide comprises the amino acid sequence of SEQ ID NO:18, or a biologically active fragment thereof, and the catalytic polypeptide comprises the amino acid sequence of SEQ ID NO:19, or a biologically active fragment thereof.

5. The recombinant polypeptide of claim 2, wherein the prodomain polypeptide comprises the amino acid sequence of SEQ ID NO:35, or a biologically active fragment thereof, and the catalytic polypeptide comprises the amino acid sequence of SEQ ID NO:36, or a biologically active fragment thereof.

6. The recombinant polypeptide of claim 2, wherein the prodomain polypeptide comprises the amino acid sequence of SEQ ID NO:52, or a biologically active fragment thereof, and the catalytic polypeptide comprises the amino acid sequence of SEQ ID NO:53, or a biologically active fragment thereof.

7. The recombinant polypeptide of any of claims 1-6, further comprising a signal peptide attached to the N-terminus of the prodomain polypeptide.

8. The recombinant polypeptide of claim 7, wherein the signal peptide comprises an amino acid sequence selected from the group consisting of SEQ ID NO:3, SEQ ID NO:20, SEQ ID NO:37, and SEQ ID NO:54.

9. The recombinant polypeptide of claim 8, wherein the signal peptide comprises the amino acid sequence of SEQ ID NO:3.

10. The recombinant polypeptide of claim 8, wherein the signal peptide comprises the amino acid sequence of SEQ ID NO:20.

11. The recombinant polypeptide of claim 8, wherein the signal peptide comprises the amino acid sequence of SEQ ID NO:37.

12. The recombinant polypeptide of claim 8, wherein the signal peptide comprises the amino acid sequence of SEQ ID NO:54.

13. The recombinant polypeptide of any one of claims 1-12, wherein the recombinant polypeptide further comprises a linker attached to the C-terminus of the prodomain polypeptide and the N-terminus of the catalytic polypeptide.

14. The recombinant polypeptide of claim 13, wherein the when the linker is attached to the C-terminus of the prodomain, at least one amino acid residue of the last ten amino acids of the C-terminus of the prodomain polypeptide is absent.

15. The recombinant polypeptide of claim 13 or claim 14, wherein the linker comprises an amino acid sequence that is cleavable by an enzyme.

16. The recombinant polypeptide of claim 15, wherein the enzyme is a protease.

17. The recombinant polypeptide of any one of claims 13-16, wherein the linker comprises an amino acid sequence selected from the group consisting of SEQ ID NO:69, SEQ ID NO:70, SEQ ID NO:71, SEQ ID NO:72, SEQ ID NO:73, SEQ ID NO:74, and SEQ ID NO:75.

18. The recombinant polypeptide of claim 1, wherein the recombinant polypeptide has an amino acid sequence of any one of SEQ ID NOS: 6-17, 23-34, 40-51, and 57-68.

19. The recombinant polypeptide of any of claims 1-18, further comprising a purification tag attached to the N-terminus of the prodomain polypeptide or signal peptide, when present, wherein the purification tag is optionally connected to the N-terminus of the prodomain polypeptide or signal peptide through a linker (e.g., an enzyme cleavable linker).

20. The recombinant polypeptide of claim 19, wherein the purification tag comprises an amino acid sequence of any one of SEQ ID NOS: 80-84.

21. A nucleic acid construct encoding a recombinant polypeptide having at least 80% sequence identity to the recombinant polypeptide of any one of claims 1-20.

22. A nucleic acid construct encoding (a) an amino acid sequence of any one of SEQ ID NOS: 1-68, (b) comprising a sequence having at least 70% sequence identity to (a) above; and/or (c) comprising a nucleotide sequence that differs from the nucleotide sequences of (a) or (b) above due to the degeneracy of the genetic code.

23. The nucleic acid construct of claim 22, wherein the amino acid sequence of (a) is any one of SEQ ID NOs: 6-17, 23-34, 40-51, and 57-68.

24. A vector comprising the nucleic acid construct of any one of claims 21-23.

25. A composition comprising the recombinant polypeptide of any one of claims 1-20 in a pharmaceutically acceptable carrier.

26. The composition of claim 25, wherein the pharmaceutically acceptable carrier comprises sterile pyrogen-free water and/or sterile pyrogen-free physiological saline solution.

27. A composition comprising the recombinant polypeptide of any one of claims 1-20 in a cosmetically acceptable carrier.

28. The composition of claim 27, wherein the composition is in the form of a spray, an emulsion, a mousse, a liquid, a cream, an oil, a lotion, an ointment, a gel or a solid.

29. A method of treating, preventing, or reducing the risk of developing muscle spasms in a subject by administering to a subject in need thereof an effective amount of the composition of claim 25 or claim 26.

30. A method of treating, preventing or reducing the risk of developing a disorder in a subject, comprising administering to a subject in need thereof an effective amount of the composition of claim 25 or claim 26, wherein the disorder is selected from the group consisting of strabismus, blepharospasm, headaches, cervical dystonia, severe primary axillary hyperhidrosis, prostatic symptoms, asthma, post stroke spasticity diabetes, cystic fibrosis, obstructive pulmonary disease, achalasia, and any combination thereof.

31. A method of treating, preventing, or reducing the risk of developing cystic fibrosis in a subject, comprising administering to a subject in need thereof an effective amount of the composition of claim 25 or claim 26.

32. A method of treating, preventing, or reducing the risk of developing a cosmetic condition in a subject, comprising administering to a subject in need thereof an effective amount of the composition of claim 27 or claim 28, wherein the cosmetic condition is selected from the group consisting of frown wrinkles, forehead wrinkles, crow's feet, nose crease wrinkles, and any combination thereof.
